(19) United States
(12) Patent Application Publication
(10) Pub. No.: US 2020/0354730 A1
(43) Pub. Date: Nov. 12, 2020

(54) Improved Yeast Polytope Vaccine Compositions And Methods

(71) Applicant: NantCell, Inc., Culver City, CA (US)

(72) Inventors: Kayvan Niazi, Culver City, CA (US); Thomas King, Culver City, CA (US)

(21) Appl. No.: 16/762,115

(22) PCT Filed: Nov. 11, 2018
(86) PCT No.: PCT/US2018/062449

§ 371 Date: May 6, 2020

Related U.S. Application Data
(60) Provisional application No. 62/590,661, filed on Nov. 27, 2017.

(51) Int. Cl. C12N 15/81; A61K 39/00

(52) U.S. Cl. CPC C12N 15/81; A61K 2039/523; A61K 39/0011

(57) ABSTRACT

Systems and methods for yeast vaccines are presented that allow for selection of tumor neoepitopes that are then used to generate a recombinant polytope for yeast expression with enhanced immunogenicity.

This application claims priority to our copending US provisional patent application with the Ser. No. 62/590,661, filed Nov. 27, 2017, which is incorporated by reference in its entirety herein.

### FIELD OF THE INVENTION

The field of the invention is compositions and methods of improved neoepitope-based immune therapeutics, especially as it relates to preparation of yeast-based cancer vaccines.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer immunotherapies targeting certain antigens common to a specific cancer have led to remarkable responses in some patients. Unfortunately, many patients failed to respond to such immunotherapy despite apparent expression of the same antigen. One possible reason for such failure could be that various effector cells of the immune system may not have been present in sufficient quantities, or may have been exhausted. Moreover, intracellular antigen processing and HLA variability among patients may have led to insufficient processing of the antigen and/or antigen display, leading to a therapeutically ineffective or lacking response.

To increase the selection of targets for immune therapy, random mutations have more recently been considered since some random mutations in tumor cells may give rise to unique tumor specific antigens (neoepitopes). As such, and at least conceptually, neoepitopes may provide a unique precision target for immunotherapy. Additionally, it has been shown that cytolytic T-cell responses can be triggered by very small quantities of peptides (e.g., Sykulev et al., Immunity, Volume 4, Issue 6, p 565-571, 1 Jun. 1996). Moreover, due to the relatively large number of mutations in many cancers, the number of possible targets is relatively high. In view of these findings, the identification of cancer neoepitopes as therapeutic targets has attracted much attention. Unfortunately, current data appear to suggest that all or almost all cancer neoepitopes are unique to a patient and specific tumor and fail to provide any specific indication as to which neoepitope may be useful for an immunotherapeutic agent that is therapeutically effective.

To overcome at least some of the problems associated with large numbers of possible targets for immune therapy, the neoepitopes can be filtered for the type of mutation (e.g., to ascertain missense or nonsense mutation), the level of transcription to confirm transcription of the mutated gene, and to confirm protein expression. Moreover, the so filtered neoepitope may be further analyzed for specific binding to the patient's HLA system as described in WO 2016/172722. Once filtered neoepitopes are identified, corresponding recombinant nucleic acids can then be prepared that can be sub-cloned for viral gene delivery and expression of the neoepitopes in infected (e.g., dendritic) cells as is taught, for example, in commonly owned PCT/US17/23894. While conceptually attractive, generation of sufficient virus quantities will often require at least several weeks, if not months. As such, therapeutic intervention will be delayed.

Thus, even though multiple methods of identification and delivery of neoepitopes to various cells are known in the art, all or almost all of them suffer from various disadvantages. Consequently, it would be desirable to have improved systems and methods for neoepitope selection and rapid production of a neoepitope vaccine that increases the likelihood of a therapeutic response in immune therapy.

### SUMMARY OF THE INVENTION

The inventive subject matter is directed to various immune therapeutic compositions and methods, and especially recombinant yeast vaccine systems, in which multiple selected neoepitopes are combined to form a rational-designed polypeptide with a leader peptide (and especially alpha factor leader) to secrete or transport the polypeptide to the periplasmic space. Expression and transport of the recombinant polypeptide to such location will advantageously increase the immunogenicity of the polypeptide, possibly to due to enhanced exposure to macrophages and dendritic cells and/or adjuvant effect.

In one aspect of the inventive subject matter, the inventors contemplate methods of generating a yeast vaccine and/or a yeast expression vector, and methods of treating a patient with recombinant yeast vaccines to treat cancer. In these methods, a recombinant nucleic acid having a sequence that encodes a polytope that is operably linked to a promoter to drive expression of the polytope is constructed. Most preferably, the polytope comprises a leader element that directs the polytope to a location selected from the group consisting of a periplasmic space, a cell wall, and an extracellular space, and further comprises a plurality of filtered neoepitope sequences.

Most typically, but not necessarily, the yeast expression vector is expression vector for S. cerevisiae, and the promoter may be a constitutive or an inducible promoter. In further preferred aspects, leader element is an alpha-factor leader, a YAP1 leader, or a p150 leader.

Where desired, the filtered neoepitope sequences are filtered by comparing tumor versus matched normal of the same patient, are filtered to have binding affinity to an MHC complex of equal or less than 200 nM, and/or are filtered against known human SNP and somatic variations. Optionally, the filtered neoepitope sequences may have an arrangement within the polytope such that the polytope has a likelihood of a presence and/or strength of hydrophobic sequences or signal peptides that is below a predetermined threshold.

Moreover, it is contemplated that the filtered neoepitope sequences will bind to MHC-I, or to MHC-II, or to MHC-I and MHC-II.

In another aspect of the inventive subject matter, the inventors contemplate a recombinant yeast expression vector for immune therapy that includes a sequence that encodes a polytope operably linked to a promoter to drive expression of the polytope. Most preferably, the polytope comprises a leader element that directs the polytope to a location selected from the group consisting of a periplasmic space, a cell wall, and an extracellular space, and further comprises a plurality of filtered neoepitope sequences.

Most typically, but not necessarily, the yeast expression vector is expression vector for S. cerevisiae, and the promoter may be a constitutive or an inducible promoter. In further preferred aspects, leader element is an alpha-factor leader, a YAP1 leader, or a p150 leader.

Where desired, the filtered neoepitope sequences are filtered by comparing tumor versus matched normal of the same patient, are filtered to have binding affinity to an MHC complex of equal or less than 200 nM, and/or are filtered against known human SNP and somatic variations. Optionally, the filtered neoepitope sequences may have an arrangement within the polytope such that the polytope has a likelihood of a presence and/or strength of hydrophobic sequences or signal peptides that is below a predetermined threshold.

Moreover, it is contemplated that the filtered neoepitope sequences will bind to MHC-I, or to MHC-II, or to MHC-I and MHC-II.

In still another aspect of the inventive subject matter, the inventors contemplate a recombinant yeast comprising the above described recombinant yeast expression vector. Preferably, the yeast is S. cerevisiae. Additionally, still another aspect of the inventive subject matter includes a pharmaceutical composition comprising the recombinant yeast that includes the recombinant yeast described above.

Still another aspect of the inventive subject matter includes use of recombinant yeast described above in the treatment of cancer or in the manufacture of a medicament for treatment of cancer.

Still another aspect of the inventive subject matter includes a method of treating an individual. In this method, the individual is inoculated with recombinant yeast, which comprises a sequence that encodes a polytope operably linked to a promoter to drive expression of the polytope. Most preferably, the polytope comprises a leader element that directs the polytope to a location selected from the group consisting of a periplasmic space, a cell wall, and an extracellular space, and further comprises a plurality of filtered neoepitope sequences.

Most typically, but not necessarily, the yeast expression vector is expression vector for S. cerevisiae, and the promoter may be a constitutive or an inducible promoter. In further preferred aspects, leader element is an alpha-factor leader, a YAP1 leader, or a p150 leader.

Where desired, the filtered neoepitope sequences are filtered by comparing tumor versus matched normal of the same patient, are filtered to have binding affinity to an MHC complex of equal or less than 200 nM, and/or are filtered against known human SNP and somatic variations. Optionally, the filtered neoepitope sequences may have an arrangement within the polytope such that the polytope has a likelihood of a presence and/or strength of hydrophobic sequences or signal peptides that is below a predetermined threshold.

Moreover, it is contemplated that the filtered neoepitope sequences will bind to MHC-I, or to MHC-II, or to MHC-I and MHC-II.

Additionally, the method may further comprise a step of using at least some of the neoepitopes in a viral vaccine. In some embodiments, the viral vaccine is an adenoviral vaccine. In other embodiments, the individual was previously inoculated with a bacterial vaccine. In such embodiments, it is preferred that the bacterial vaccine contained a tumor associated antigen or at least of the neoepitopes.

Various objects, features, aspects and advantages of the inventive subject matter will become more apparent from the following detailed description of preferred embodiments, along with the accompanying drawing in which like numerals represent like components.

### BRIEF DESCRIPTION OF THE DRAWING

FIG. 1 is a schematic representation of various arrangements of neoepitopes in a polytope.

FIG. 2 is a schematic representation of various sequence arrangements in a polytope.

FIG. 3 shows exemplary arrangements of neoepitopes in polytopes using alpha factor leader sequences (shown as SEQ ID. No. 1-12).

### DETAILED DESCRIPTION

The inventors have discovered that neoepitope-based immune therapy can be further improved by providing a recombinant yeast vaccine to a patient, typically before the patient receives an adenovirus-based vaccine, wherein the yeast and the adenovirus vaccine have most preferably the same neoepitopes. As such, the yeast vaccine can be administered in at least some cases as a prime vaccine while the adenoviral vaccine can be given as a boost vaccine. For particularly effective yeast vaccine formulations, the inventors contemplate that the yeast is transfected with a recombinant nucleic acid, from which one or more neoepitopes (e.g., as polytope) are expressed as fusion proteins with a leader sequence that directs the polypeptide to the periplamic space (and in some cases beyond the periplamic space).

Viewed from a different perspective, it should be appreciated that the compositions and methods presented herein will include one or more neoepitopes that are specific to the patient and the tumor in the patient to allow for targeted treatment. Moreover, such treatment may advantageously be tailored to achieve one or more specific immune reactions, including a CD4⁺ biased immune response, a CD8⁺ biased immune response, antibody biased immune response, and/or a stimulated immune response (e.g., reducing checkpoint inhibition and/or by activation of immune competent cells using cytokines). Most typically, such effects are in achieved in the context of the neoepitopes originating from the recombinant nucleic acid.

Neoepitopes can be characterized as expressed random mutations in tumor cells that created unique and tumor specific antigens. Therefore, viewed from a different perspective, neoepitopes may be identified by considering the type (e.g., deletion, insertion, transversion, transition, translocation) and impact of the mutation (e.g., non-sense, missense, frame shift, etc.), which may as such serve as a content filter through which silent and other non-relevant (e.g., non-expressed) mutations are eliminated. It should also be appreciated that neoepitope sequences can be defined as sequence stretches with relatively short length (e.g., 8-12 mers or 14-20mers) wherein such stretches will include the change(s) in the amino acid sequences. Most typically, but not necessarily, the changed amino acid will be at or near the central amino acid position. For example, a typical neoepitope may have the structure of A₄-N-A₄, or A₃-N-A₅, or A₂-N-A₇, or A₅-N-A₃, or A₇-N-A₂, where A is a proteinogenic wild type or normal (i.e., from corresponding healthy tissue of the same patient) amino acid and N is a changed amino acid (relative to wild type or relative to matched normal). Therefore, the neoepitope sequences contemplated herein include sequence stretches with relatively short length (e.g., 5-30 mers, more typically 8-12 mers, or 14-20 mers) wherein such stretches include the change(s) in the amino acid sequences. Where desired, additional amino acids may be placed upstream or downstream of the changed amino acid, for example, to allow for additional antigen processing in the various compartments (e.g., for proteasome processing in the cytosol, or specific protease processing in the endosomal and/or lysosomal compartments) of a cell.

Thus, it should be appreciated that a single amino acid change may be presented in numerous neoepitope sequences that include the changed amino acid, depending on the position of the changed amino acid. Advantageously, such sequence variability allows for multiple choices of neoepitopes and as such increases the number of potentially useful targets that can then be selected on the basis of one or more desirable traits (e.g., highest affinity to a patient HLA-type, highest structural stability, etc.). Most typically, neoepitopes will be calculated to have a length of between 2-50 amino acids, more typically between 5-30 amino acids, and most typically between 8-12 amino acids, or 14-20 amino acids, with the changed amino acid preferably centrally located or otherwise situated in a manner that improves its binding to MHC. For example, where the epitope is to be presented by the MHC-I complex, a typical neoepitope length will be about 8-12 amino acids, while the typical neoepitope length for presentation via MHC-II complex will have a length of about 14-20 amino acids. As will be readily appreciated, since the position of the changed amino acid in the neoepitope may be other than central, the actual peptide sequence and with that actual topology of the neoepitope may vary considerably, and the neoepitope sequence with a desired binding affinity to the MHC-I or MHC-II presentation and/or desired protease processing will typically dictate the particular sequence.

Of course, it should be appreciated that the identification or discovery of neoepitopes may start with a variety of biological materials, including fresh biopsies, frozen, or otherwise preserved tissue or cell samples, circulating tumor cells, exosomes, various body fluids (and especially blood), etc. Therefore, suitable methods of omics analysis include nucleic acid sequencing, and particularly NGS methods operating on DNA (e.g., Illumina sequencing, ion torrent sequencing, 454 pyrosequencing, nanopore sequencing, etc.), RNA sequencing (e.g., RNAseq, reverse transcription based sequencing, etc.), and in some cases protein sequencing or mass spectroscopy based sequencing (e.g., SRM, MRM, CRM, etc.).

As such, and particularly for nucleic acid based sequencing, it should be particularly recognized that high-throughput genome sequencing of a tumor tissue will allow for rapid identification of neoepitopes. However, it must be appreciated that where the so obtained sequence information is compared against a standard reference, the normally occurring inter-patient variation (e.g., due to SNPs, short indels, different number of repeats, etc.) as well as heterozygosity will result in a relatively large number of potential false positive neoepitopes. Notably, such inaccuracies can be eliminated where a tumor sample of a patient is compared against a matched normal (i.e., non-tumor) sample of the same patient.

In one especially preferred aspect of the inventive subject matter, DNA analysis is performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination of the same patient. Therefore, data sets suitable for use herein include unprocessed or processed data sets, and exemplary preferred data sets include those having BAM format, SAM format, GAR format, FASTQ format, or FASTA format, as well as BAMBAM, SAMBAM, and VCF data sets. However, it is especially preferred that the data sets are provided in BAM format or as BAMBAM diff objects as is described in US2012/0059670A1 and US2012/0066001A1. Moreover, it should be noted that the data sets are reflective of a tumor and a matched normal sample of the same patient. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor and/or metastatic site, etc. In most cases, the matched normal sample of the patient is blood, or a non-diseased tissue from the same tissue type as the tumor.

Likewise, the computational analysis of the sequence data may be performed in numerous manners. In most preferred methods, however, analysis is performed in silico by location-guided synchronous alignment of tumor and normal samples as, for example, disclosed in US 2012/0059670 and US 2012/0066001 using BAM files and BAM servers. Such analysis advantageously reduces false positive neoepitopes and significantly reduces demands on memory and computational resources.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Viewed from a different perspective, a patient- and cancer-specific in silico collection of sequences can be established that encode neoepitopes having a predetermined length of, for example, between 5 and 25 amino acids and include at least one changed amino acid. Such collection will typically include for each changed amino acid at least two, at least three, at least four, at least five, or at least six members in which the position of the changed amino acid is not identical. Such collection advantageously increases potential candidate molecules suitable for immune therapy and can then be used for further filtering (e.g., by sub-cellular location, transcription/expression level, MHC-I and/or II affinity, etc.) as is described in more detail below.

For example, and using synchronous location guided analysis to tumor and matched normal sequence data, the inventors previously identified various cancer neoepitopes from a variety of cancers and patients, including the following cancer types: BLCA, BRCA, CESC, COAD, DLBC, GBM, HNSC, KICH, KIRC, KIRP, LAML, LGG, LIHC, LUAD, LUSC, OV, PRAD, READ, SARC, SKCM, STAD, THCA, and UCEC. Exemplary neoepitope data for these cancers can be found in International application PCT/US16/29244, incorporated by reference herein.

Depending on the type and stage of the cancer, as well as the patient's immune status, it should be recognized that not all of the identified neoepitopes will necessarily lead to a therapeutically equally effective reaction in a patient. Indeed, it is well known in the art that only a fraction of neoepitopes will generate an immune response. To increase likelihood of a therapeutically desirable response, the initially identified neoepitopes can be further filtered. Of course, it should be appreciated that downstream analysis need not take into account silent mutations for the purpose of the methods presented herein. However, preferred mutation analyses will provide in addition to the particular type of mutation (e.g., deletion, insertion, transversion, transition, translocation) also information of the impact of the mutation (e.g., non-sense, missense, etc.) and may as such serve as a first content filter through which silent mutations are eliminated. For example, neoepitopes can be selected for further consideration where the mutation is a frame-shift, non-sense, and/or missense mutation.

In a further filtering approach, neoepitopes may also be subject to detailed analysis for sub-cellular location parameters. For example, neoepitope sequences may be selected for further consideration if the neoepitopes are identified as having a membrane associated location (e.g., are located at the outside of a cell membrane of a cell) and/or if an in silico structural calculation confirms that the neoepitope is likely to be solvent exposed, or presents a structurally stable epitope (e.g., J Exp Med 2014), etc.

With respect to filtering neoepitopes, it is generally contemplated that neoepitopes are especially suitable for use herein where omics (or other) analysis reveals that the neoepitope is actually expressed. Identification of expression and expression level of a neoepitope can be performed in all manners known in the art and preferred methods include quantitative RNA (hnRNA or mRNA) analysis and/or quantitative proteomics analysis. Most typically, the threshold level for inclusion of neoepitopes will be an expression level of at least 20%, at least 30%, at least 40%, or at least 50% of expression level of the corresponding matched normal sequence, thus ensuring that the (neo)epitope is at least potentially ‘visible’ to the immune system. Consequently, it is generally preferred that the omics analysis also includes an analysis of gene expression (transcriptomic analysis) to so help identify the level of expression for the gene with a mutation.

There are numerous methods of transcriptomic analysis known in the art, and all of the known methods are deemed suitable for use herein. For example, preferred materials include mRNA and primary transcripts (hnRNA), and RNA sequence information may be obtained from reverse transcribed polyAtRNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNAseq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

In yet another aspect of filtering, the neoepitopes may be compared against a database that contains known human sequences (e.g., of the patient or a collection of patients) to so avoid use of a human-identical sequence. Moreover, filtering may also include removal of neoepitope sequences that are due to SNPs in the patient where the SNPs are present in both the tumor and the matched normal sequence. For example, dbSNP (The Single Nucleotide Polymorphism Database) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation. Using such database and other filtering options as described above, the patient and tumor specific neoepitopes may be filtered to remove those known sequences, yielding a sequence set with a plurality of neoepitope sequences having substantially reduced false positives.

Once the desired level of filtering for the neoepitope is accomplished (e.g., neoepitope filtered by tumor versus normal, and/or expression level, and/or sub-cellular location, and/or patient specific HLA-match, and/or known variants), a further filtering step is contemplated that takes into account the gene type that is affected by the neoepitope. For example, suitable gene types include cancer driver genes, genes associated with regulation of cell division, genes associated with apoptosis, and genes associated with signal transduction. However, in especially preferred aspects, cancer driver genes are particularly preferred (which may span by function a variety of gene types, including receptor genes, signal transduction genes, transcription regulator genes, etc.). In further contemplated aspects, suitable gene types may also be known passenger genes and genes involved in metabolism.

With respect to the identification or other determination (e.g., prediction) of a gene as being a cancer driver gene, various methods and prediction algorithms are known in the art, and are deemed suitable for use herein. For example, suitable algorithms include MutsigCV (Nature 2014, 505(7484):495-501), ActiveDriver (Mol Syst Biol 2013, 9:637), MuSiC (Genome Res 2012, 22(8):1589-1598), OncodriveClust (Bioinformatics 2013, 29(18):2238-2244), OncodriveFM (Nucleic Acids Res 2012, 40(21):e169), OncodriveFML (Genome Biol 2016, 17(1):128), Tumor Suppressor and Oncogenes (TUSON) (Cell 2013, 155(4):948-962), 20/20+(https://github.com/KarchinLab/2020plus), and oncodriveROLE (Bioinformatics (2014) 30 (17): i549-i555). Alternatively, or additionally, identification of cancer driver genes may also employ various sources for known cancer driver genes and their association with specific cancers. For example, the Intogen Catalog of driver mutations (2016.5; URL: www.intogen.org) contains the results of the driver analysis performed by the Cancer Genome Interpreter across 6,792 exomes of a pan-cancer cohort of 28 tumor types.

Nevertheless, despite filtering, it should be recognized that not all neoepitopes will be visible to the immune system as the neoepitopes also need to be processed where present in a larger context (e.g., within a polytope) and presented on the MHC complex of the patient. In that context, it must be appreciated that only a fraction of all neoepitopes will have sufficient affinity for presentation. Consequently, and especially in the context of immune therapy it should be apparent that neoepitopes will be more likely effective where the neoepitopes are properly processed, bound to, and presented by the MHC complexes. Viewed from another perspective, treatment success will be increased with an increasing number of neoepitopes that can be presented via the MHC complex, wherein such neoepitopes have a minimum affinity to the patient's HLA-type. Consequently, it should be appreciated that effective binding and presentation is a combined function of the sequence of the neoepitope and the particular HLA-type of a patient. Therefore, HLA-type determination of the patient tissue is typically required. Most typically, the HLA-type determination includes at least three MHC-I sub-types (e.g., HLA-A, HLA-B, HLA-C, etc.) and at least three MHC-II sub-types (e.g., HLA-DP, HLA-DQ, HLA-DR, etc.), preferably with each subtype being determined to at least 2-digit or at least 4-digit depth. However, greater depth (e.g., 6 digit, 8 digit, etc.) is also contemplated.

Once the HLA-type of the patient is ascertained (using known chemistry or in silico determination), a structural solution for the HLA-type is calculated and/or obtained from a database, which is then used in a docking model in silico to determine binding affinity of the (typically filtered) neoepitope to the HLA structural solution. As will be further discussed below, suitable systems for determination of binding affinities include the NetMHC platform (see e.g., Nucleic Acids Res. 2008 Jul. 1; 36(Web Server issue): W509-W512.). Neoepitopes with high affinity (e.g., less than 100 nM, less than 75 nM, less than 50 nM) for a previously determined HLA-type are then selected for therapy creation, along with the knowledge of the patient's MHC-I/II subtype.

HLA determination can be performed using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types. For example, in one preferred method according to the inventive subject matter, a relatively large number of patient sequence reads mapping to chromosome 6p21.3 (or any other location near/at which HLA alleles are found) is provided by a database or sequencing machine. Most typically the sequence reads will have a length of about 100-300 bases and comprise metadata, including read quality, alignment information, orientation, location, etc. For example, suitable formats include SAM, BAM, FASTA, GAR, etc. While not limiting to the inventive subject matter, it is generally preferred that the patient sequence reads provide a depth of coverage of at least 5×, more typically at least 10×, even more typically at least 20×, and most typically at least 30×.

In addition to the patient sequence reads, contemplated methods further employ one or more reference sequences that include a plurality of sequences of known and distinct HLA alleles. For example, a typical reference sequence may be a synthetic (without corresponding human or other mammalian counterpart) sequence that includes sequence segments of at least one HLA-type with multiple HLA-alleles of that HLA-type. For example, suitable reference sequences include a collection of known genomic sequences for at least 50 different alleles of HLA-A. Alternatively, or additionally, the reference sequence may also include a collection of known RNA sequences for at least 50 different alleles of HLA-A. Of course, and as further discussed in more detail below, the reference sequence is not limited to 50 alleles of HLA-A, but may have alternative composition with respect to HLA-type and number/composition of alleles. Most typically, the reference sequence will be in a computer readable format and will be provided from a database or other data storage device. For example, suitable reference sequence formats include FASTA, FASTQ, EMBL, GCG, or GenBank format, and may be directly obtained or built from data of a public data repository (e.g., IMGT, the International ImMunoGeneTics information system, or The Allele Frequency Net Database, EUROSTAM, URL: www.allelefrequencies.net). Alternatively, the reference sequence may also be built from individual known HLA-alleles based on one or more predetermined criteria such as allele frequency, ethnic allele distribution, common or rare allele types, etc.

Using the reference sequence, the patient sequence reads can now be threaded through a de Bruijn graph to identify the alleles with the best fit. In this context, it should be noted that each individual carries two alleles for each HLA-type, and that these alleles may be very similar, or in some cases even identical. Such high degree of similarity poses a significant problem for traditional alignment schemes. The inventor has now discovered that the HLA alleles, and even very closely related alleles can be resolved using an approach in which the de Bruijn graph is constructed by decomposing a sequence read into relatively small k-mers (typically having a length of between 10-20 bases), and by implementing a weighted vote process in which each patient sequence read provides a vote (“quantitative read support”) for each of the alleles on the basis of k-mers of that sequence read that match the sequence of the allele. The cumulatively highest vote for an allele then indicates the most likely predicted HLA allele. In addition, it is generally preferred that each fragment that is a match to the allele is also used to calculate the overall coverage and depth of coverage for that allele.

Scoring may further be improved or refined as needed, especially where many of the top hits are similar (e.g., where a significant portion of their score comes from a highly shared set of k-mers). For example, score refinement may include a weighting scheme in which alleles that are substantially similar (e.g., >99%, or other predetermined value, etc.) to the current top hit are removed from future consideration. Counts for k-mers used by the current top hit are then re-weighted by a factor (e.g., 0.5, etc.), and the scores for each HLA allele are recalculated by summing these weighted counts. This selection process is repeated to find a new top hit. The accuracy of the method can be even further improved using RNA sequence data that allows identification of the alleles expressed by a tumor, which may sometimes be just 1 of the 2 alleles present in the DNA. In further advantageous aspects of contemplated systems and methods, DNA or RNA, or a combination of both DNA and RNA can be processed to make HLA predictions that are highly accurate and can be derived from tumor or blood DNA or RNA. Further aspects, suitable methods and considerations for high-accuracy in silico HLA typing are described in WO 2017/035392, incorporated by reference herein.

Once patient and tumor specific neoepitopes and HLA-type are identified, further computational analysis can be performed by in silico docking neoepitopes to the HLA and determining best binders (e.g., lowest K_{D}, for example, less than 500 nM, or less than 250 nM, or less than 150 nM, or less than 50 nM, etc.), for example, using NetMHC. It should be appreciated that such approach will not only identify specific neoepitopes that are genuine to the patient and tumor, but also those neoepitopes that are most likely to be presented on a cell and as such most likely to elicit an immune response with therapeutic effect. Of course, it should also be appreciated that thusly identified HLA-matched neoepitopes can be biochemically validated in vitro prior to inclusion of the nucleic acid encoding the epitope as payload into the virus as is further discussed below.

Of course, it should be appreciated that matching of the patient's HLA-type to the patient- and cancer-specific neoepitope can be done using systems other than NetMHC, and suitable systems include NetMHC II, NetMHCpan, IEDB Analysis Resource (URL immuneepitope.org), RankPep, PREDEP, SVMHC, Epipredict, HLABinding, and others (see e.g., J Immunol Methods 2011; 374:1-4). In calculating the highest affinity, it should be noted that the collection of neoepitope sequences in which the position of the altered amino acid is moved (supra) can be used. Alternatively, or additionally, modifications to the neoepitopes may be implemented by adding N- and/or C-terminal modifications to further increase binding of the expressed neoepitope to the patient's HLA-type. Thus, neoepitopes may be native as identified or further modified to better match a particular HLA-type. Moreover, where desired, binding of corresponding wild type sequences (i.e., neoepitope sequence without amino acid change) can be calculated to ensure high differential affinities. For example, especially preferred high differential affinities in MHC binding between the neoepitope and its corresponding wild type sequence are at least 2-fold, at least 5-fold, at least 10-fold, at least 100-fold, at least 500-fold, at least 1000-fold, etc.).

Binding affinity, and particularly differential binding affinity may also be determined in vitro using various systems and methods. For example, antigen presenting cells of a patient or cells with matched HLA-type can be transfected with a nucleic acid (e.g., viral, plasmid, linear DNA, RNA, etc.) to express one or more neoepitopes using constructs as described in more detail below. Upon expression and antigen processing, the neoepitopes can then be identified in the MHC complex on the outside of the cell, either using specific binders to the neoepitope or using a cell based system (e.g., PBMC of the patient, etc.) in which T cell activation or cytotoxic NK cell activity can be observed in vitro. Neoepitopes with differential activity (elicit a stronger signal or immune response as compared to the corresponding wild type epitope) will then be selected for therapy creation.

Upon identification of desired neoepitopes, one or more recombinant yeast immune vaccine compositions may be prepared using the sequence information of the neoepitopes. Among other yeast strains, it is especially preferred that the patient may be treated with a recombinant Saccharomyces train that is genetically modified with a nucleic acid construct as further discussed below that leads to expression of at least one of the identified neoepitopes to thereby initiate an immune response against the tumor. Any yeast strain can be used to produce a yeast vehicle of the present invention. Yeast are unicellular microorganisms that belong to one of three classes: Ascomycetes, Basidiomycetes and Fungi Imperfecti. One consideration for the selection of a type of yeast for use as an immune modulator is the pathogenicity of the yeast. In preferred embodiments, the yeast is a non-pathogenic strain such as Saccharomyces cerevisiae as non-pathogenic yeast strains minimize any adverse effects to the individual to whom the yeast vehicle is administered. However, pathogenic yeast may also be used if the pathogenicity of the yeast can be negated using pharmaceutical intervention.

For example, suitable genera of yeast strains include Saccharomyces, Candida, Cryptococcus, Hansenula, Kluyveromyces, Pichia, Rhodotorula, Schizosaccharomyces and Yarrowia. In one aspect, yeast genera are selected from Saccharomyces, Candida, Hansenula, Pichia or Schizosaccharomyces, and in a preferred aspect, Saccharomyces is used. Species of yeast strains that may be used in the invention include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica.

It should further be appreciated that a number of these species include a variety of subspecies, types, subtypes, etc. that are intended to be included within the aforementioned species. In one aspect, yeast species used in the invention include S. cerevisiae, C. albicans, H. polymorpha, P. pastoris and S. pombe. S. cerevisiae is useful due to it being relatively easy to manipulate and being “Generally Recognized As Safe” or “GRAS” for use as food additives (GRAS, FDA proposed Rule 62FR18938, Apr. 17, 1997). Therefore, the inventors particularly contemplate a yeast strain that is capable of replicating plasmids to a particularly high copy number, such as a S. cerevisiae cir strain. The S. cerevisiae strain is one such strain that is capable of supporting expression vectors that allow one or more target antigen(s) and/or antigen fusion protein(s) and/or other proteins to be expressed at high levels. In addition, any mutant yeast strains can be used in the present invention, including those that exhibit reduced post-translational modifications of expressed target antigens or other proteins, such as mutations in the enzymes that extend N-linked glycosylation.

Expression of contemplated neoepitopes in yeast can be accomplished using techniques known to those skilled in the art. Most typically, a nucleic acid molecule encoding at least neoepitope or other protein is inserted into an expression vector such manner that the nucleic acid molecule is operatively linked to a transcription control sequence to be capable of effecting either constitutive or regulated expression of the nucleic acid molecule when transformed into a host yeast cell. As will be readily appreciated, nucleic acid molecules encoding one or more antigens and/or other proteins can be on one or more expression vectors operatively linked to one or more expression control sequences. Particularly important expression control sequences are those which control transcription initiation, such as promoter and upstream activation sequences.

Any suitable yeast promoter can be used in the present invention and a variety of such promoters are known to those skilled in the art. Promoters for expression in Saccharomyces cerevisiae include, but are not limited to, promoters of genes encoding the following yeast proteins: alcohol dehydrogenase I (ADH1) or II (ADH2), CUP1, phosphoglycerate kinase (PGK), triose phosphate isomerase (TPI), translational elongation factor EF-1 alpha (TEF2), glyceraldehyde-3-phosphate dehydrogenase (GAPDH; also referred to as TDH3, for triose phosphate dehydrogenase), galactokinase (GAL1), galactose-1-phosphate uridyl-transferase (GAL7), UDP-galactose epimerase (GAL10), cytochrome c1 (CYC1), Sec7 protein (SECT) and acid phosphatase (PHO5), including hybrid promoters such as ADH2/GAPDH and CYC1/GAL10 promoters, and including the ADH2/GAPDH promoter, which is induced when glucose concentrations in the cell are low (e.g., about 0.1 to about 0.2 percent), as well as the CUP1 promoter and the TEF2 promoter. Likewise, a number of upstream activation sequences (UASs), also referred to as enhancers, is known. Upstream activation sequences for expression in Saccharomyces cerevisiae include the UASs of genes encoding the following proteins: PCK1, TPI, TDH3, CYC1, ADH1, ADH2, SUC2, GAL1, GAL7 and GAL10, as well as other UASs activated by the GAL4 gene product, with the ADH2 UAS being used in one aspect. Since the ADH2 UAS is activated by the ADR1 gene product, it may be preferable to overexpress the ADR1 gene when a heterologous gene is operatively linked to the ADH2 UAS. Transcription termination sequences for expression in Saccharomyces cerevisiae include the termination sequences of the alpha-factor, GAPDH, and CYC1 genes. Transcription control sequences to express genes in methyltrophic yeast include the transcription control regions of the genes encoding alcohol oxidase and formate dehydrogenase.

Likewise, transfection of a nucleic acid molecule into a yeast cell according to the present invention can be accomplished by any method by which a nucleic acid molecule administered into the cell and includes diffusion, active transport, bath sonication, electroporation, microinjection, lipofection, adsorption, and protoplast fusion. Transfected nucleic acid molecules can be integrated into a yeast chromosome or maintained on extrachromosomal vectors using techniques known to those skilled in the art. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using techniques known to those skilled in the art to produce cytoplast, ghost or subcellular yeast membrane extract or fractions thereof containing desired antigens or other proteins. Further exemplary yeast expression systems, methods, and conditions are described in US 2012/0107347.

In this context, it should be appreciated that the manner of neoepitope arrangement and rational-designed trafficking of the neoepitopes can have a substantial impact on the efficacy of various immune therapeutic compositions. For example, single neoepitopes can be expressed individually from the respective recombinant constructs that are delivered as a single plasmid, viral expression construct, etc. Alternatively, multiple neoepitopes can be separately expressed from individual promoters to form individual mRNA that are then individually translated into the respective neoepitopes, or from a single mRNA comprising individual translation starting points for each neoepitope sequence (e.g., using 2A or IRES signals). Notably, while such arrangements are generally thought to allow for controlled delivery of proper neoepitope peptide, efficacy of such expression systems has been less than desirable (data not shown).

In contrast, where multiple neoepitopes were expressed from a single transcript to so form a single transcript that is then translated into a single polytope (i.e., polypeptide with a series of concatemerically linked neoepitopes, optionally with intervening linker sequences) expression, processing, and antigen presentation was found to be effective. Notably, the expression of polytopes requires processing by the appropriate proteases (e.g., proteasome, endosomal proteases, lysosomal proteases) within a cell to yield the neoepitope sequences, and polytopes led to improved antigen processing and presentation for most neoepitopes as compared to expression of individual neoepitopes, particularly where the individual neoepitopes had a relatively short length (e.g., less than 25 amino acids; results not shown). Moreover, such approach also allows rational design of protease sensitive sequence motifs between the neoepitope peptide sequences to so assure or avoid processing by specific proteases as the proteasome, endosomal proteases, and lysosomal proteases have distinct cleavage preferences. Therefore, polytopes may be designed that include not only linker sequences to spatially separate neoepitopes, but also sequence portions (e.g., between 3-15 amino acids) that will be preferentially cleaved by a specific protease.

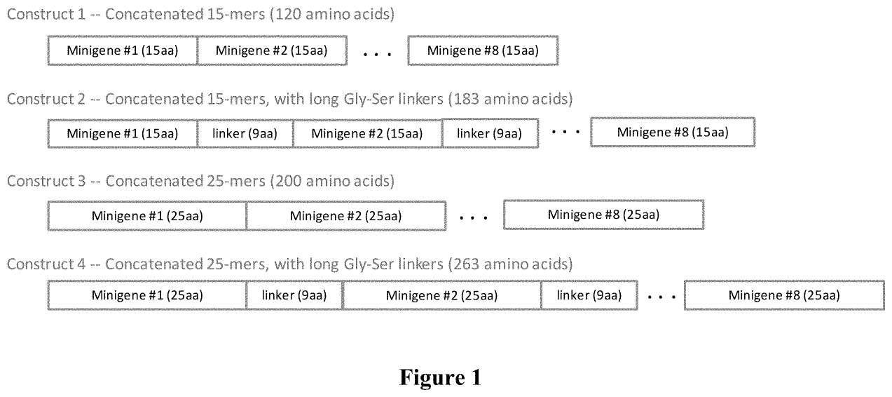

Therefore, the inventors contemplate recombinant nucleic acids and yeast expression vectors that comprise a nucleic acid segment that encodes a polytope wherein the polytope is operably coupled to a desired promoter element, and wherein individual neoepitopes are optionally separated by a linker and/or protease cleavage or recognition sequence. For example, FIG. 1 exemplarily illustrates various contemplated arrangements for neoepitopes for expression from yeast expression system. Here, Construct 1 exemplarily illustrates a neoepitope arrangement that comprises eight neoepitopes (‘minigene’) with a total length of 15 amino acids in concatemeric series without intervening linker sequences, while Construct 2 shows the arrangement of Construct 1 but with inclusion of nine amino acid linkers between each neoepitope sequence. Of course, and as already noted above, it should be recognized that the exact length of the neoepitope sequence is not limited to 15 amino acids, and that the exact length may vary considerably. However, in most cases, where neoepitope sequences of between 8-12 amino acids are flanked by additional amino acids, the total length will typically not exceed 25 amino acids, or 30 amino acids, or 50 amino acids. Likewise, it should be noted that while FIG. 1 denotes G-S linkers, various other linker sequences are also suitable for use herein. Such relatively short neoepitopes are especially beneficial where presentation of the neoepitope is intended to be via the MHC-I complex.

In this context, it should be appreciated that suitable linker sequences will provide steric flexibility and separation of two adjacent neoepitopes. However, care must be taken to as to not choose amino acids for the linker that could be immunogenic/form an epitope that is already present in a patient. Consequently, it is generally preferred that the polytope construct is filtered once more for the presence of epitopes that could be found in a patient (e.g., as part of normal sequence or due to SNP or other sequence variation). Such filtering will apply the same technology and criteria as already discussed above.

Similarly, Construct 3 exemplarily illustrates a neoepitope arrangement that includes eight neoepitopes in concatemeric series without intervening linker sequences, and Construct 4 shows the arrangement of Construct 3 with inclusion of nine amino acid linkers between each neoepitope sequence. As noted above, it should be recognized that the exact length of such neoepitope sequences is not limited to 25 amino acids, and that the exact length may vary considerably. However, in most cases, where neoepitope sequences of between 14-20 amino acids are flanked by additional amino acids, the total length will typically not exceed 30 amino acids, or 45 amino acids, or 60 amino acids. Likewise, it should be noted that while FIG. 1 denotes G-S linkers for these constructs, various other linker sequences are also suitable for use herein. Such relatively long neoepitopes are especially beneficial where presentation of the neoepitope is intended to be via the MHC-II complex.

In this example, it should be appreciated that the 15-amino acid minigenes are MHC Class I targeted tumor mutations selected with 7 amino acids of native sequence on either side, and that the 25-amino acid minigenes are MHC Class II targeted tumor mutations selected with 12 amino acids of native sequence on either side. The exemplary 9 amino acid linkers are deemed to have sufficient length such that “unnatural” MHC Class I epitopes will not form between adjacent minigenes. Polytope sequences tended to be processed and presented more efficiently than single neoepitopes (data not shown), and addition of amino acids beyond 12 amino acids for MHC-I presentation and addition of amino acids beyond 20 amino acids for MHC-I presentation appeared to allow for somewhat improved protease processing.

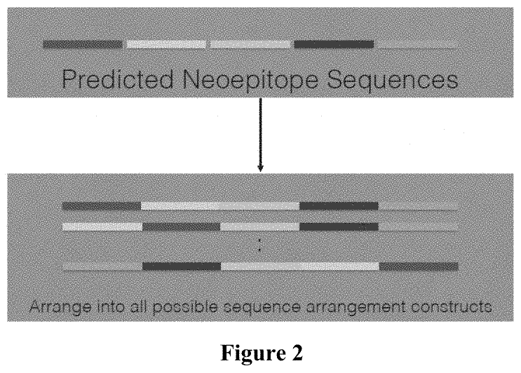

To maximize the likelihood that customized protein sequences are properly processed for presentation by the HLA complex, neoepitope sequences may be arranged in a manner to minimize hydrophobic sequences that may result in immediate trafficking to the cell membrane or into the extracellular space. Most preferably, hydrophobic sequence or signal peptide detection is done either by comparison of sequences to a weight matrix (see e.g., Nucleic Acids Res. 1986 Jun. 11; 14(11): 4683-4690) or by using neural networks trained on peptides that contain signal sequences (see e.g., Journal of Molecular Biology 2004, Volume 338, Issue 5, 1027-1036). FIG. 2 depicts an exemplary scheme of arrangement selection in which a plurality of polytope sequences is analyzed. Here, all positional permutations of all neoepitopes are calculated to produce a collection of arrangements. This collection is then processed through a weight matrix and/or neural network prediction to generate a score representing the likelihood of presence and/or strength of hydrophobic sequences or signal peptides. All positional permutations are then ranked by score, and the permutation(s) with a score below a predetermined threshold or lowest score for likelihood of presence and/or strength of hydrophobic sequences or signal peptides is/are used to construct a customized neoepitope expression cassette.

With respect to the total number of neoepitope sequences in a polytope, it is generally preferred that the polytope comprise at least two, or at least three, or at least five, or at least eight, or at least ten neoepitope sequences. Indeed, the payload capacity of the recombinant DNA is generally contemplated the limiting factor, along with the availability of filtered and appropriate neoepitopes.

Regardless of the particular arrangement of the neoepitope sequences, it is generally contemplated that each polytope or neoepitope has a leader or other signaling sequence that prompts translocation of the polytope or neoepitope across the plasma membrane into the periplasmic space, cell wall, and/or across the cell wall. Therefore, in particularly preferred aspects, the leader sequence may be derived from the alpha-factor of S. cerevisiae and may include the entire pre-sequence, or portions thereof. For example, particularly suitable sequence arrangements are described in U.S. Pat. No. 7,198,919. However, shorter sequence portions are also deemed suitable for use herein.

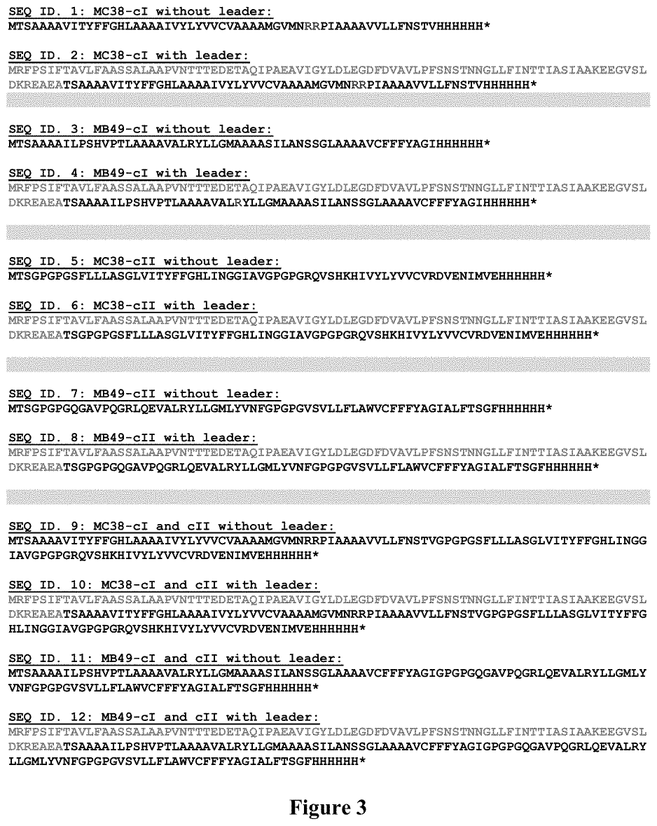

FIG. 3 provides an exemplary set of recombinant polypeptides (shown as SEQ ID. No. 1-12) resulting from the expression of the corresponding recombinant nucleic acids. More particularly, neoepitopes for MC38 colon cancer cells and MB49 urothelial carcinoma cells were determined as noted above and nucleic acids were constructed with linker sequences between the neoepitopes. Neoepitopes for class I presentation are designated cI, while neoepitopes for class II presentation are designated cII. Leader sequences are indicated in red. As discussed above, and as shown in FIG. 3, it should be recognized that neoepitopes can be directed to class I presentation, class II presentation, or both.

While not limiting to the inventive subject matter, it is contemplated that transport to the periplasmic space (and even cell wall) will provide an enhancement of immune stimulation, possibly due to adjuvant effect of cell wall components, and/or early exposure of the expressed neoepitopes to the antigen presenting cells/macrophages. Of course, it should be noted that while alpha-factor leader sequences are especially preferred, other leader sequences from S.cerevisiae and other yeast are also deemed suitable for use herein, and include the p150 leader, the Exp1 leader, and the YAP1 leader (e.g., Nature Biotechnology 8, 42-46 (1990)).

Upon transfection and expression of the various neoepitopes and/or polytopes in the yeast, the recombinant yeast can then be further processed to form a yeast vaccine as a medicament for treatment of cancer, for example, by formulating the transfected yeast in a pharmaceutically acceptable carrier, typically following protocols well known in the art.

The inventors contemplate that such generated recombinant yeast or yeast vaccine carrying the recombinant nucleic acids can be used to induce or generate antigen presenting cells (e.g., dendritic cells) in vivo or ex vivo to express the chimeric protein and the tumor-associated antigen to enhance the immune response against the tumor cell expressing the tumor-associated antigen. Thus, in some embodiments, one or more recombinant yeast including one or more nucleic acid segments encoding the chimeric protein and/or one or more tumor-associated antigen, cytokine, and/or co-stimulatory molecule can be administered to the patient to infect antigen presenting cells in vivo. Such infected antigen presenting cells are expected to express one or more tumor-associated antigen, cytokine, and/or co-stimulatory molecules to so stimulate immune response against the tumor cells by simulating CD40 signaling, activating antigen presenting cells, and further activating immune competent cells, preferably T cells, interacting such activated antigen presenting cells.

For example, a recombinant yeast or yeast vaccine that carries the recombinant nucleic acid encoding the chimeric protein and/or one or more tumor-associated antigen can be formulated in any pharmaceutically acceptable carrier (e.g., preferably formulated as a sterile injectable composition, etc.) to form a pharmaceutical composition. The recombinant yeast or yeast vaccine can be administered to the patient, or the patient can be inoculated with the recombinant yeast or yeast vaccine, in any suitable methods. In some embodiments, where a cytokine (e.g., ALT-805) is desired to be expressed in the same cell, it is contemplated that the recombinant nucleic acid of the recombinant yeast or yeast vaccine further includes a nucleic acid encoding the cytokine, or that another recombinant yeast including a recombinant nucleic acid encoding the cytokine can be generated. Where two or more types of the recombinant yeasts are desired to infect the same antigen presenting cell, it is preferred that the two or more types of the recombinant yeasts can be formulated in a single pharmaceutical composition. However, it is also contemplated that two or more types of the recombinant yeasts are formulated in two separate and distinct pharmaceutical compositions and administered to the patient concurrently or substantially concurrently (e.g., within an hour, within 2 hours, within a day, etc.).

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). In some embodiments, the yeast formulation is administered via systemic injection including subcutaneous, subdermal injection, or intravenous injection. In other embodiments, where the systemic injection may not be efficient (e.g., for brain tumors, etc.), it is contemplated that the formulation is administered via intratumoral injection. Alternatively, or additionally, antigen presenting cells may be isolated or grown from cells of the patient, infected in vitro, and then transfused to the patient. Therefore, it should be appreciated that contemplated systems and methods can be considered a complete drug discovery system (e.g., drug discovery, treatment protocol, validation, etc.) for highly personalized cancer treatment.

With respect to dose and schedule of the formulation administration, it is contemplated that the dose and/or schedule may vary depending on depending on the type of yeast, type and prognosis of disease (e.g., tumor type, size, location), health status of the patient (e.g., including age, gender, etc.). While it may vary, the dose and schedule may be selected and regulated so that the formulation does not provide any significant toxic effect to the host normal cells, yet sufficient to be elicit an immune response. Thus, in a preferred embodiment, an optimal or desired condition of administering the formulation can be determined based on a predetermined threshold. For example, the predetermined threshold may be a predetermined local or systemic concentration of specific type of cytokine (e.g., IFN-γ, TNF-β, IL-2, IL-4, IL-10, etc.). Therefore, administration conditions are typically adjusted to have immune response-specific cytokines expressed at least 20%, at least 30%, at least 50%, at least 60%, at least 70% more at least locally or systemically.

In some embodiments, the administration of the pharmaceutical formulation can be in two or more different stages: a priming administration and a boost administration; or a first-stage administration and a second-stage administration. Thus, the inventors contemplate that different types of vaccines can be used as a priming administration and a boost administration considering their difference in multiplication cycle and expression speed. Preferably, such different types of vaccines may include viral vaccine or bacterial vaccine that includes a recombinant nucleic acid encoding a tumor associated antigen and/or a neoepitope. More preferably, the tumor associated antigen and/or a neoepitope encoded by the recombinant nucleic acid of the viral vaccine or bacterial vaccine is the same or substantially similar to those encoded by the polytope of the recombinant yeast, such that two types of vaccines can elicit the immune response against the same or substantially similar molecule. For example, it is contemplated that the patient is administered a viral vaccine (e.g., adenoviral vaccine) as a priming administration (or the first-stage administration) and the yeast vaccine as a boost administration (or the second-stage administration) at least 3 days, at least 5 days, at least 7 days, at least 2 weeks after the priming administration. Alternatively, the yeast vaccine can be administered as a priming administration (or the first-stage administration) and the viral vaccine as a boost administration (or the second-stage administration) at least 3 days, at least 5 days, at least 7 days, at least 2 weeks after the priming administration. In another example, it is contemplated that the patient is administered a bacteria vaccine as a priming administration (or the first-stage administration) and the yeast vaccine as a boost administration (or the second-stage administration) at least 3 days, at least 5 days, at least 7 days, at least 2 weeks after the priming administration. Alternatively, the yeast vaccine can be administered as a priming administration (or the first-stage administration) and the bacteria vaccine as a boost administration (or the second-stage administration) at least 3 days, at least 5 days, at least 7 days, at least 2 weeks after the priming administration.

Where desired, additional therapeutic modalities may be employed which may be neoepitope based (e.g., synthetic antibodies against neoepitopes as described in WO 2016/172722), alone or in combination with autologous or allogenic NK cells, and especially haNK cells or taNK cells (e.g., both commercially available from NantKwest, 9920 Jefferson Blvd. Culver City, Calif. 90232). Where haNK or taNK cells are employed, it is particularly preferred that the haNK cell carries a recombinant antibody on the CD16 variant that binds to a neoepitope of the treated patient, and where taNK cells are employed it is preferred that the chimeric antigen receptor of the taNK cell binds to a neoepitope of the treated patient. The additional treatment modality may also be independent of neoepitopes, and especially preferred modalities include cell-based therapeutics such as activated NK cells (e.g., aNK cells, commercially available from NantKwest, 9920 Jefferson Blvd. Culver City, Calif. 90232), and non cell-based therapeutics such as chemotherapy and/or radiation. In still further contemplated aspects, immune stimulatory cytokines, and especially IL-2, IL15, and IL-21 may be administered, alone or in combination with one or more checkpoint inhibitors (e.g., ipilimumab, nivolumab, etc.). Similarly, it is still further contemplated that additional pharmaceutical intervention may include administration of one or more drugs that inhibit immune suppressive cells, and especially MDSCs Tregs, and M2 macrophages. Thus, suitable drugs include IL-8 or interferon-γ inhibitors or antibodies binding IL-8 or interferon-γ, as well as drugs that deactivate MDSCs (e.g., NO inhibitors, arginase inhibitors, ROS inhibitors), that block development of or differentiation of cells to MDSCs (e.g., IL-12, VEGF-inhibitors, bisphosphonates), or agents that are toxic to MDSCs (e.g., gemcitabine, cisplatin, 5-FU). Likewise, drugs like cyclophosphamide, daclizumab, and anti-GITR or anti-OX40 antibodies may be used to inhibit Tregs.

Alternatively and/or additionally, non-host cells (e.g., bacteria cells) can be co-administered with the recombinant yeast or yeast vaccine to boost the immune response. For example, contemplated bacterial cells include those modified to have no or reduced expression of expresses lipopolysaccharides that would otherwise trigger an immune response and cause endotoxic responses, which can lead potentially fatal sepsis (e.g., CD-14 mediated sepsis). Thus, one exemplary bacteria strain with modified lipopolysaccharides includes ClearColi® BL21(DE3) electrocompetent cells. This bacteria strain is BL21 with a genotype F-ompT hsdSB (rB-mB) gal dcm lonλ(DE3 [lacI lacUV5-T7 gene 1 ind1 sam7 nin5]) msbA148 ΔgutQΔkdsD ΔlpxLΔlpxMΔpagPΔlpxPΔeptΔ. In this context, it should be appreciated that several specific deletion mutations (ΔgutQ ΔkdsD ΔlpxL ΔlpxMΔpagP ΔlpxP ΔeptA) encode the modification of LPS to Lipid IVA, while one additional compensating mutation (msbA148) enables the cells to maintain viability in the presence of the LPS precursor lipid IVA. These mutations result in the deletion of the oligosaccharide chain from the LPS. More specifically, two of the six acyl chains are deleted. The six acyl chains of the LPS are the trigger which is recognized by the Toll-like receptor 4 (TLR4) in complex with myeloid differentiation factor 2 (MD-2), causing activation of NF-κB and production of proinflammatory cytokines. Lipid IVA, which contains only four acyl chains, is not recognized by TLR4 and thus does not trigger the endotoxic response. While electrocompetent BL21 bacteria is provided as an example, the inventors contemplates that the genetically modified bacteria can be also chemically competent bacteria.

Alternatively, an inactive or weakened bovine tuberculosis bacillus strain (e.g., Bacillus Calmette-Guérin (BCG) vaccine) can be used as an adjuvant. Further, the inventors also contemplate that the patient's own endosymbiotic bacteria can be used as a non-host cell. As used herein, the patient's endosymbiotic bacteria refers bacteria residing in the patient's body regardless of the patient's health condition without invoking any substantial immune response. Thus, it is contemplated that the patient's endosymbiotic bacteria is a normal flora of the patient. For example, the patient's endosymbiotic bacteria may include E. coli or Streptococcus that can be commonly found in human intestine or stomach. In these embodiments, patient's own endosymbiotic bacteria can be obtained from the patient's biopsy samples from a portion of intestine, stomach, oral mucosa, or conjunctiva, or in fecal samples. The patient's endosymbiotic bacteria can then be cultured in vitro and transfected with nucleotides encoding human disease-related antigen(s). In still further contemplated aspects, the bacterial non-host cell may also be a pathogenic cell, including Bordetella pertussis and/or Mycobacterium bovis. Most typically, but not necessarily, the bacterial non-host cells will be killed before exposure to the host cells.

Nonpathogenic yeast cells may be co-administered with the recombinant yeast or yeast vaccine to boost the immune response as well. There are numerous yeast strains suitable for use herein, and most typically non-pathogenic yeasts include Saccharomyces cerevisiae, Saccharomyces boulardi, Pichia pasteuris, Schizosaccharomyces pombe, Candida stellata, etc. As noted above, such yeast strains may be further genetically modified to reduce one or more adverse traits, and/or to express a recombinant protein that further increases yeast infectivity and/or expression. Contemplated yeast strains are typically commercially available and can be modified using protocols well known in the art. While not limiting the inventive subject matter by any particular theory or hypothesis, the inventors contemplate that one or more components of the non-host cells may act as a danger or damage signal, particularly where the host cells are immune competent cells. Therefore, the inventors not only contemplate use of non-host cells per se, but also one or more immune stimulating portions thereof. Therefore, especially contemplated portions include ligands for PAMP receptors, ligands for DAMP receptors, TLR ligands, CpG, ssDNA, and thapsigargin.

To trigger overexpression or transcription of stress signals, it is also contemplated that the chemotherapy and/or radiation for the patient may be done using a low-dose regimen, preferably in a metronomic fashion. For example, it is generally preferred that such treatment will use doses effective to affect at least one of protein expression, cell division, and cell cycle, preferably to induce apoptosis or at least to induce or increase the expression of stress-related genes (and particularly NKG2D ligands). Thus, in further contemplated aspects, such treatment will include low dose treatment using one or more chemotherapeutic agents. Most typically, low dose treatments will be at exposures that are equal or less than 70%, equal or less than 50%, equal or less than 40%, equal or less than 30%, equal or less than 20%, equal or less than 10%, or equal or less than 5% of the LD₅₀ or IC₅₀ for the chemotherapeutic agent. Additionally, where advantageous, such low-dose regimen may be performed in a metronomic manner as described, for example, in U.S. Pat. Nos. 7,758,891, 7,771,751, 7,780,984, 7,981,445, and 8,034,375.

With respect to the particular drug used in such low-dose regimen, it is contemplated that all chemotherapeutic agents are deemed suitable. Among other suitable drugs, kinase inhibitors, receptor agonists and antagonists, anti-metabolic, cytostatic and cytotoxic drugs are all contemplated herein. However, particularly preferred agents include those identified to interfere or inhibit a component of a pathway that drives growth or development of the tumor. Suitable drugs can be identified using pathway analysis on omics data as described in, for example, WO 2011/139345 and WO 2013/062505. Most notably, so achieved expression of stress-related genes in the tumor cells will result in surface presentation of NKG2D, NKP30, NKP44, and/or NKP46 ligands, which in turn activate NK cells to specifically destroy the tumor cells. Thus, it should be appreciated that low-dose chemotherapy may be employed as a trigger in tumor cells to express and display stress related proteins, which in turn will trigger NK-cell activation and/or NK-cell mediated tumor cell killing. Additionally, NK-cell mediated killing will be associated with release of intracellular tumor specific antigens, which is thought to further enhance the immune response.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of generating a yeast expression vector for immune therapy, the method comprising:
constructing a recombinant nucleic acid having a sequence that encodes a polytope that is operably linked to a promoter to drive expression of the polytope;
wherein the polytope comprises a leader element that directs the polytope to a location selected from the group consisting of a periplasmic space, a cell wall, and an extracellular space; and
wherein the polytope comprises a plurality of filtered neoepitope sequences.

2. The method of claim 1, wherein the yeast expression vector is expression vector for Saccharomyces cerevisiae.

3. The method of claim 1, wherein the promoter is a constitutive promoter.

4. The method of claim 1, wherein the promoter is an inducible promoter.

5-12. (canceled)

13. The method of claim 1, wherein the leader element is selected from the group consisting of an alpha-factor leader, a YAP1 leader, and a p150 leader.

14. The method of claim 1, wherein the filtered neoepitope sequences are filtered by comparing tumor versus matched normal of the same patient.

15. The method of claim 1, wherein the filtered neoepitope sequences are filtered to have binding affinity to an MHC complex of equal or less than 200 nM.

16. The method of claim 1, wherein the filtered neoepitope sequences are filtered against known human SNP and somatic variations.

17. The method of claim 1, wherein the filtered neoepitope sequences have an arrangement within the polytope such that the polytope has a likelihood of a presence and/or strength of hydrophobic sequences or signal peptides that is below a predetermined threshold.

18. The method of claim 1, wherein the filtered neoepitope sequences bind to MHC-I.

19. The method of claim 1, wherein the filtered neoepitope sequences bind to MHC-II.

20. The method of claim 1, wherein the filtered neoepitope sequences bind to MHC-I and MHC-II.

21. A recombinant yeast expression vector for immune therapy, comprising:
a sequence that encodes a polytope operably linked to a promoter to drive expression of the polytope;
wherein the polytope comprises a leader element that directs the polytope to a location selected from the group consisting of a periplasmic space, a cell wall, and an extracellular space; and
wherein the polytope comprises a plurality of filtered neoepitope sequences.

22. The yeast expression vector 21, wherein the yeast expression vector is expression vector for S. cerevisiae.

23. The yeast expression vector 21, wherein the promoter is a constitutive promoter.

24. The yeast expression vector 21, wherein the promoter is an inducible promoter.

25-33. (canceled)

34. The yeast expression vector of claim 21, wherein the leader element is selected from the group consisting of an alpha-factor leader, a YAP1 leader, and a p150 leader.

35. The yeast expression vector of claim 21, wherein the filtered neoepitope sequences are filtered by comparing tumor versus matched normal of the same patient.

36. The yeast expression vector of claim 21, wherein the filtered neoepitope sequences are filtered to have binding affinity to an MHC complex of equal or less than 200 nM.

37-46. (canceled)

47. A method of treating an individual, the method comprising:
inoculating the individual with a recombinant yeast;
wherein the recombinant yeast comprises a sequence that encodes a polytope operably linked to a promoter to drive expression of the polytope;
wherein the polytope comprises a leader element that directs the polytope to a location selected from the group consisting of a periplasmic space, a cell wall, and an extracellular space; and
wherein the polytope comprises a plurality of filtered neoepitope sequences.

48-62. (canceled)


SEQUENCE LISTING (12 sequences)

SEQ ID NO: 1 - protein, 61 aa, Artificial Sequence
  MC38-cI without leader
MTSAAAAVITYFFGHLAAAAIVYLYVVCVAAAAMGVMNRRPIAAAAVVLLFNSTVHHHHHH

SEQ ID NO: 2 - protein, 149 aa, Artificial Sequence
  MC38-cI with leader
MRFPSIFTAVLFAASSALAAPVNTTTEDETAQIPAEAVIGYLDLEGDFDVAVLPFSNSTNNGLLFINTTIASIAAKEEGVSLDKREAEATSAAAAVITYFFGHLAAAAIVYLYVVCVAAAAMGVMNRRPIAAAAVVLLFNSTVHHHHHH

SEQ ID NO: 3 - protein, 61 aa, Artificial Sequence
  MB49-cI without leader
MTSAAAAILPSHVPTLAAAAVALRYLLGMAAAASILANSSGLAAAAVCFFFYAGIHHHHHH

SEQ ID NO: 4 - protein, 149 aa, Artificial Sequence
  MB49-cI with leader
MRFPSIFTAVLFAASSALAAPVNTTTEDETAQIPAEAVIGYLDLEGDFDVAVLPFSNSTNNGLLFINTTIASIAAKEEGVSLDKREAEATSAAAAILPSHVPTLAAAAVALRYLLGMAAAASILANSSGLAAAAVCFFFYAGIHHHHHH

SEQ ID NO: 5 - protein, 69 aa, Artificial Sequence
  MC38-cII without leader
MTSGPGPGSFLLLASGLVITYFFGHLINGGIAVGPGPGRQVSHKHIVYLYVVCVRDVENIMVEHHHHHH

SEQ ID NO: 6 - protein, 157 aa, Artificial Sequence
  MC38-cII with leader
MRFPSIFTAVLFAASSALAAPVNTTTEDETAQIPAEAVIGYLDLEGDFDVAVLPFSNSTNNGLLFINTTIASIAAKEEGVSLDKREAEATSGPGPGSFLLLASGLVITYFFGHLINGGIAVGPGPGRQVSHKHIVYLYVVCVRDVENIMVEHHHHHH

SEQ ID NO: 7 - protein, 69 aa, Artificial Sequence
  MB49-cII without leader
MTSGPGPGQGAVPQGRLQEVALRYLLGMLYVNFGPGPGVSVLLFLAWVCFFFYAGIALFTSGFHHHHHH

SEQ ID NO: 8 - protein, 157 aa, Artificial Sequence
  MB49-cII with leader
MRFPSIFTAVLFAASSALAAPVNTTTEDETAQIPAEAVIGYLDLEGDFDVAVLPFSNSTNNGLLFINTTIASIAAKEEGVSLDKREAEATSGPGPGQGAVPQGRLQEVALRYLLGMLYVNFGPGPGVSVLLFLAWVCFFFYAGIALFTSGFHHHHHH

SEQ ID NO: 9 - protein, 121 aa, Artificial Sequence
  MC38-cI and cII without leader
MTSAAAAVITYFFGHLAAAAIVYLYVVCVAAAAMGVMNRRPIAAAAVVLLFNSTVGPGPGSFLLLASGLVITYFFGHLINGGIAVGPGPGRQVSHKHIVYLYVVCVRDVENIMVEHHHHHH

SEQ ID NO: 10 - protein, 209 aa, Artificial Sequence
  MC38-cI and cII with leader
MRFPSIFTAVLFAASSALAAPVNTTTEDETAQIPAEAVIGYLDLEGDFDVAVLPFSNSTNNGLLFINTTIASIAAKEEGVSLDKREAEATSAAAAVITYFFGHLAAAAIVYLYVVCVAAAAMGVMNRRPIAAAAVVLLFNSTVGPGPGSFLLLASGLVITYFFGHLINGGIAVGPGPGRQVSHKHIVYLYVVCVRDVENIMVEHHHHHH

SEQ ID NO: 11 - protein, 121 aa, Artificial Sequence
  MB49-cI and cII without leader
MTSAAAAILPSHVPTLAAAAVALRYLLGMAAAASILANSSGLAAAAVCFFFYAGIGPGPGQGAVPQGRLQEVALRYLLGMLYVNFGPGPGVSVLLFLAWVCFFFYAGIALFTSGFHHHHHH

SEQ ID NO: 12 - protein, 209 aa, Artificial Sequence
  MB49-cI and cII with leader
MRFPSIFTAVLFAASSALAAPVNTTTEDETAQIPAEAVIGYLDLEGDFDVAVLPFSNSTNNGLLFINTTIASIAAKEEGVSLDKREAEATSAAAAILPSHVPTLAAAAVALRYLLGMAAAASILANSSGLAAAAVCFFFYAGIGPGPGQGAVPQGRLQEVALRYLLGMLYVNFGPGPGVSVLLFLAWVCFFFYAGIALFTSGFHHHHHH